(19) United States
(12) Patent Application Publication
(10) Pub. No.: US 2005/0239073 A1
(43) Pub. Date: Oct. 27, 2005

(54) Fluorescent entity comprising a fluorophore covalently attached to at least one oligonucleotide and comprising at least one functional group, and uses thereof

(76) Inventors: Eric Trinquet, Pont Saint Esprit (FR); Fabrice Maurin, Saint Nazaire (FR); Herve Bazin, Villeneuve les Avignon (FR); Gerard Mathis, Bagnols sur Ceze (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/516,744

(22) PCT Filed: Jun. 6, 2003
(86) PCT No.: PCT/EP03/06459

§ 371 Date: Dec. 6, 2004

(30) Foreign Application Priority Data
Jun. 6, 2002 (FR) 02/06948

(51) Int. Cl. C12Q 1/68; C07H 21/04

(52) U.S. Cl. 435/6; 536/25.32

(57) ABSTRACT

The invention relates to a fluorescent entity comprising a fluorophore, with the exception of a rare earth metal cryptate, covalently attached to one or more oligonucleotide(s) or oligonucleotide analog(s) and comprising at least one functional group, introduced or generated on the fluorophore or one of the oligonucleotides or oligonucleotide analogs, allowing it to be coupled with a carrier molecule. The invention also relates to a fluorescent conjugate consisting of a fluorescent entity covalently attached to a carrier molecule.

The invention relates to a fluorescent entity comprising a fluorophore, covalently attached to one or more oligo-nucleotides or oligonucleotide analogs and comprising at least one functional group, introduced or generated on the fluorophore or one of the oligonucleotides or oligonucleotide analogs.

Many families of organic molecules are used as fluorescent labels for biomolecules in a number of applications, in particular for diagnostic methods which make it possible to follow or to quantify these biomolecules.

Mention may in particular be made of rhodamines, cyanins, squaraines or bodipy dyes. Most of these molecules have a high molar extinction coefficient (often greater than 100 000 M¹ cm¹) and a quantum yield of fluorescence generally greater than 20%.

However, due to their often very hydrophobic nature, these organic molecules have a tendency to form aggregates at the surface of biomolecules, in particular proteins, when the desire is to have several fluorescent labels per protein (U. Schobel et al., Bioconjugate chem., 1999, 10, 1107-1114). Since these aggregates are virtually nonfluorescent, the mean quantum yield of the fluorescent molecules present at the surface of the proteins is then significantly lower than that of the native molecules.

To overcome this problem, an attempt has been made, in the literature, to increase the hydrophilic nature of the fluorescent molecules, in particular by adding sulfonate groups (U.S. Pat. No. 5,268,486). Mention may also be made of the addition of sugars or of carbohydrate residues to the structure of the fluorescent molecules (WO 98/49176). However, the addition of these units limits the aggregation phenomenon without suppressing it.

Other approaches have also been used to avoid the decrease in quantum yield of fluorescence after labeling of proteins. Application WO 98/26287 describes a method using cyclodextrins to encapsulate fluorescent molecules at the surface of proteins and to limit their aggregation. However, this technique does not function satisfactorily with all types of molecules.

A technique has recently been described which makes it possible to obtain proteins having, at their surface, only a single fluorescent molecule and makes it possible to thus be able to maintain a high quantum yield by avoiding the aggregation phenomenon (Winckler et al., Specific labeling of proteins using reactive affinity tag-dye systems, SBS Conference, Vancouver, 2000). This system is limiting since it does not make it possible to have several fluorescent molecules per protein.

The problem to be solved therefore consists in providing a label which can be attached to a biomolecule, the photophysical properties of which are not modified, in particular by aggregation phenomena, when several of these labels are simultaneously attached to a biomolecule, in particular a protein.

According to the invention, this problem can be solved by attaching the fluorophore to one or more oligo-nucleotide(s) or oligonucleotide analog(s), the compound thus formed also comprising one or more reactional group(s) allowing it to be attached to a carrier molecule.

According to a first aspect, the invention therefore relates to a fluorescent entity comprising a fluorophore, with the exception of a rare earth metal cryptate, covalently attached to one or more oligonucleotide(s) or oligonucleotide analog(s), characterized in that it comprises at least one functional group, introduced or generated on the fluorophore or on one of the oligo-nucleotides or oligonucleotide analogs.

The fluorophore of the entity according to the invention preferably comprises one or more aromatic rings and has a high molecular extinction coefficient, greater than 20 000, preferably greater than 50 000.

Said fluorophore is preferably chosen from rhodamines, cyanins, squaraines, bodipys, fluoresceines and their derivatives.

The term “oligonucleotide” denotes equally oligodeoxyribonucleotides (DNA fragment) or oligoribonucleotides (RNA fragment).

The term “oligonucleotide or oligonucleotide analog” is intended to mean, in the present description:
either a series of ribonucleotide or deoxyribonucleotide units attached to one another via bonds of the phosphodiester type;
or a series of ribonucleotide or deoxyribonucleotide units or of nucleotide analog units modified on the sugar or on the base and attached to one another by natural internucleotide bonds of the phosphodiester type, some of the internucleotide bonds being optionally replaced with phosphonate, phosphoramide or phosphorothioate bonds. These various oligo-nucleotide families are described in Goodchild, Bioconjugate Chemistry, 1(3), May/June 1990, 77-99;
or a series comprising both ribonucleotide or deoxyribonucleotide units attached to one another by bonds of the phosphodiester type and nucleoside analog units attached to one another by amide bonds, commonly called “PNAs” (“peptide nucleic acids”), as described in M. Egholm et al., J. Am. Chem. Soc., 1992, 114, 1895-1897; such compounds are, for example, described in R. Vinayak et al., Nucleoside & Nucleotide, 1997, 16 (7-9), 1653-1656;
or a series of ribonucleotide or deoxyribonucleotide units in which some of the nucleosides or of the internucleotide bonds have been modified compared to a natural oligoribonucleotide or oligodeoxyribonucleotide formed from the common nucleosides (adenosine, deoxyadenosine, cytidine, deoxycytidine, guanosine, deoxyguanosine, uridine or thymidine) attached by phosphate bridges, for example a phosphorothioate oligonucleotide (OPT) in which all or part of the phosphate bridges have been replaced with thiophosphate bridges (P. J. Romaniuk, F. Eckstein (1982) J. Biol. Chem. 257, 7684).

The use of each one of these types of oligonucleotide constitutes an advantageous aspect of the invention.

The term nucleotide or nucleoside “analog” is intended to mean a nucleotide/nucleoside comprising at least one modification relating to the sugar or the nucleobase or a combination of these modifications. By way of example, mention may be made of the following modifications:

I. Modifications Concerning the Sugar (Nucleotide or Nucleoside Analogs):

1°) The sugar component can be modified in that the configuration of the hydroxyls (free or involved in a phosphate bridge) is different from the natural configuration (which is, respectively, β-D-erythro in the DNA series and β-D-ribo in the RNA series), as in the analogs having the backbone β-D-arabino-pentofuranoside or β-D-xylo-pentofuranoside, for example.

20°) The structure can be modified in that the internucleotide bonds are of the 2′→5′ type, such as in the case of β-D-ribo-pentofuranoside-2′-phosphate or 3′-deoxy-β-D-erythro-pentofuranoside-2′-phosphate derivatives.

Nucleotides exist in which the structure includes the preceding two modifications, such as β-D-xylo-pentofuranoside-2′-phosphate.

3°) The structure can differ from the natural model in that the 4′ carbon has the opposite configuration, this is the case α-L-threo-pentofuranoside-3′-phosphate. The difference may relate to the configuration of the carbon in the 1′-position (anomeric position), this is the case of α-D-erythro-pentofuranoside-3′-phosphate. Nucleotides/nucleosides exist in which the structure includes the preceding two modifications, such as β-L-threo-pentofuranoside-3′-phosphate.

4°) The structure can differ from the natural model in that the oxygen in the 4′-position is replaced with a carbon (carbocyclic analog) or with a sulfur, such as 4′-thio-β-D-erythro-pentafuranoside-3′-phosphate.

5°) The structure can differ from the natural model in that one of the hydroxyls of the sugar is alkylated, for example in the backbone 2′-O-alkyl-β-D-ribo-pentafuranoside-3′-phosphate, the alkyl group possibly being, for example, the methyl or allyl group.

6°) The structure can differ from the natural model in that only the sugar component is conserved, such as in 1,2-dideoxy-D-erythro-pentafuranose-3-phosphate, or in that the sugar is replaced with a polyol such as propanediol.

II. Modifications Concerning the Nucleobase (Nucleotide Analogs):

1°) The nucleobase can be modified in that the substituents of the natural bases are modified, such as in 2,6-diaminopurine, hypoxanthine, 4-thiothymine, 4-thiouracil or 5-ethynyluracil.

2°) The positions of the substituents can be switched compared to the natural bases, such as in isoguanosine or isocytosine.

3°) A nitrogen atom of the nucleobase can be replaced with a carbon, as in the 7-deazaguanosine or 7-deazaadenine.

III. Modifications Concerning the Internucleotide Bond:

Moreover, as mentioned above, the bonds between the sugar units or their analogs can also be modified, for example by replacing one or more of the oxygen atoms of the natural phosphodiester bond with a carbon (phosphonate series), a nitrogen (phosphoramide series) or a sulfur (phosphorothioates).

The internucleotide bonds can also be replaced with amide bonds, as in oligonucleotide analogs of the “PNA” type.

According to a preferred aspect, the oligonucleotide consists of a series comprising both ribonucleotide or deoxyribonucleotide units attached to one another by bonds of the phosphodiester type and nucleoside analog units attached to one another by amide bonds.

In particular, in this case, said oligonucleotide can comprise at least five internucleotide bonds of the phosphodiester type at the end intended to be attached to the fluorophore.

Preferably, said oligonucleotide or oligonucleotide analog comprises from 5 to 60 nucleotide units, in particular from 5 to 20, preferably 5 to 15 nucleotide units.

The fluorescent entity according to the invention should comprise at least one functional group which allows it to be coupled with a carrier molecule.

Advantageously, the functional group is an amine function of a nucleotide unit of the oligonucleotide or of the oligonucleotide analog, or results from the reaction of a free amine function of a nucleotide unit of the oligonucleotide or of the oligonucleotide analog using a homobifunctional or heterobifunctional reagent which makes it possible to introduce a functional group chosen from the groups: activated ester of a carboxylic acid, carboxylic acid, isothiocyanate, aldehyde, carbonyl, sulfonyl halide, alkyl halide, azide, hydrazide, dichlorotriazine, anhydride, haloacetamide, maleimide and sulfhydryl. The homobifunctional and heterobifunctional reagents and also their use are described in “Bioconjugation” (chapters 5.3 to 5.6, M. Aslam & A. Dent, Macmillan, London, 1998).

Said functional group can, for example, result from the reaction of a free amine function of a nucleotide unit of the oligonucleotide or of the oligonucleotide analog, with an N-hydroxysuccinimidyl ester.

According to a preferred aspect, the functional group is chosen from the groups: maleimide, carboxylic acid, haloacetamide, alkyl halide, azido, hydrazido, aldehyde, ketone, amino, sulfhydryl, isothiocyanate, isocyanate, monochlorotriazine, dichlorotriazine, aziridine, sulfonyl halide, acid halide, hydroxysuccinimide ester, hydroxy-sulfosuccinimide ester, imido ester, hydrazide, azido-nitrophenyl, azidophenyl, azide, 3-(2-pyridyldithio)-proprionamide and glyoxal, and more particularly the groups of formula: 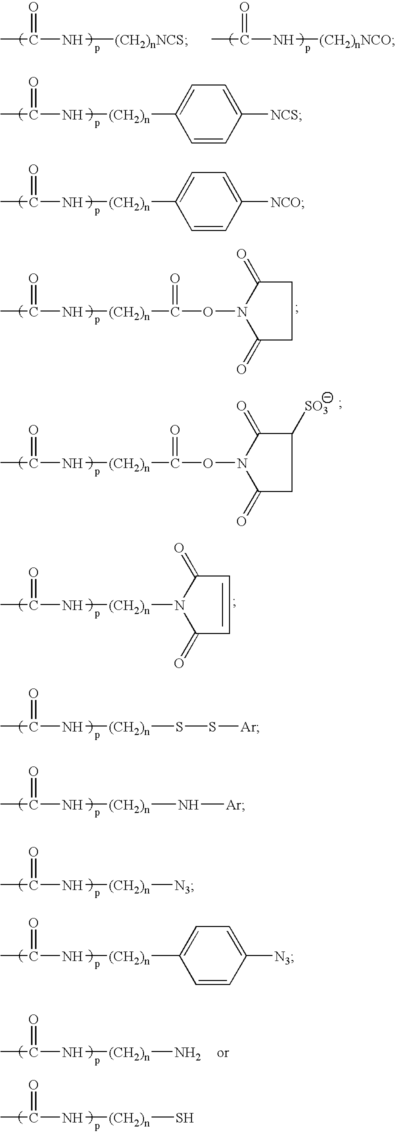
where n ranges from 0 to 8 and p is equal to 0 or 1, and Ar is a 5- or 6-membered heterocycle comprising 1 to 3 hetero atoms, optionally substituted with a halogen atom.

According to an advantageous aspect, the functional group(s) is (are) attached to the fluorophore and/or to the oligonucleotide by a spacer arm consisting of a divalent organic radical, chosen from linear or branched C₁-C₂₀ alkylene groups optionally containing one or more double bonds or triple bonds and/or optionally containing one or more hetero atoms, such as oxygen, nitrogen, sulfur, phosphorus, or one or more carbamoyl or carboxamido group(s); C₅-C₈ cycloalkylene groups and C₆-C₁₄ arylene groups, said alkylene, cycloalkylene or arylene groups being optionally substituted with alkyl, aryl or sulfonate groups.

In particular, the spacer arm is chosen from the groups: 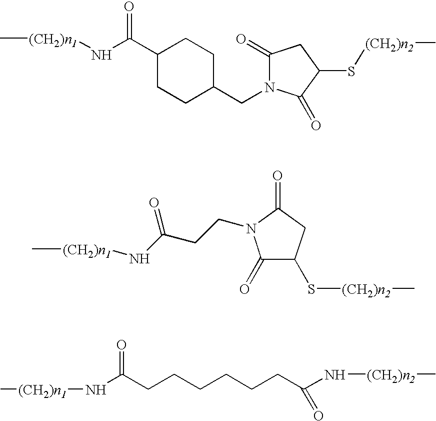
in which n₁ and n₂ are between 2 and 6.

According to a preferred aspect, the invention relates to a fluorescent entity of formula (I) 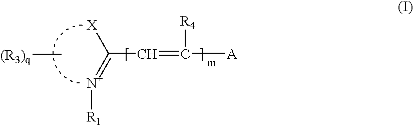
in which:
A represents a group chosen from: 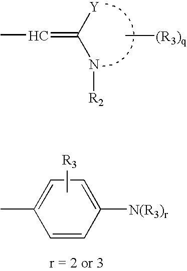 —N(R₃)ᵣ
r=2 or 0.3
the dashed lines each represent the carbon atoms required to form 1 to 3 fused rings, the groups R₃ being attached to these rings;
X and Y each represent N, C═O, O, S or C(CH₃)₂
m has a value 1, 2, 3 or 4;
q has a value 1, 2 or 3;
(R₃)_{q} represents q groups R₃, which may be identical or different;
the groups R₁, R₂ and R₃ are identical or different and are chosen from hydrogen; a group —(CH₂)ₛ-Z in which s ranges from 0 to 4 and Z represents a group CH₃, SO₃H, OH or N⁺R₁R₂R₃ in which R₁, R₂ and R₃ are as defined above; a functional group as defined above; and an oligonucleotide or oligonucleotide analog optionally comprising a functional group as defined above;
R₄ is chosen from: H; OH; CH₃; Cl and the groups of formula: 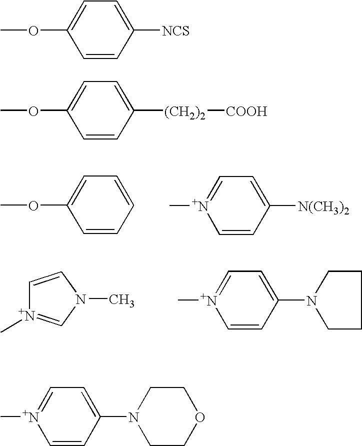
the substituents R₄ in the allylic position possibly forming, with the polyethylenic chain, 1 to 3 fused rings containing from 4 to 14 atoms, which may or may not be saturated, said rings possibly containing one or more atoms of O, N and S, and possibly being optionally substituted with an oxo group.

Preferred fluorescent entities according to the invention correspond to formulae (II) and (III) 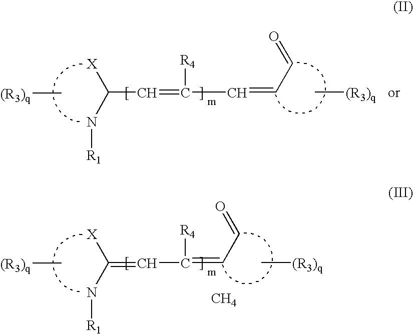
in which the dashed lines, R₁, R₂, R₃, R₄, X, m and q are as defined above for formula (I).

Advantageously, the invention relates to fluorescent entities of formula (IV), (V), (VI) or (VII) 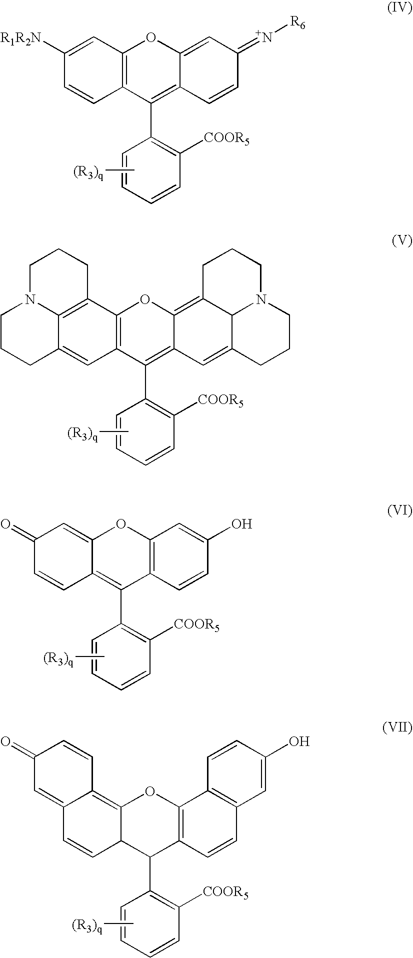
in which R₁, R₂, R₃ and R₅ are identical or different and are chosen from hydrogen; a group —(CH₂)ₛ-Z in which s ranges from 0 to 4 and Z represents a group CH₃, SO₃H, OH or N⁺R₁R₂R₃ in which R₁, R₂ and R₃ are as defined above; a functional group or an oligonucleotide or oligonucleotide analog as defined above.

According to another preferred aspect, the invention relates to a fluorescent entity of formula (VIII) 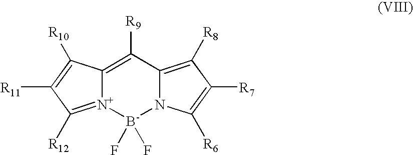
in which the substituents R₆ to R₁₂ are chosen from: hydrogen; a halogen; an alkyl; a cycloalkyl; aryl; arylalkyl; acyl; sulfo; a functional group or an oligonucleotide or oligonucleotide analog as defined above.

Another fluorescent entity according to the invention corresponds to formula (IX) 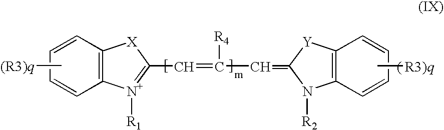
in which R₁, R₂, R₃, R₄, X, Y, m and q are as defined above.

Entities of formula (IX) which are particularly preferred are those in which X and Y represent a group C(CH₃)₂, and also those in which
R₁ and R₂ represent an alkyl comprising from 1 to 4 carbon atoms or a group of formula below, at least one of the groups R₁ and R₂ representing a group of formula below: 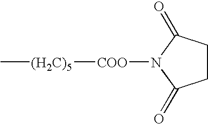
R₄ represents hydrogen
q=1, m=2

R₃ represents hydrogen; a group —(CH₂)ₛ-Z in which s ranges from 0 to 4 and Z represents a group CH₃, SO₃H, OH or N⁺R₁R₂R₃ in which R₁, R₂ and R₃ are as defined above; a functional group or an oligonucleotide or oligonucleotide analog as defined above;
R₄ is chosen from: H; OH; CH₃; Cl and the groups of formula: 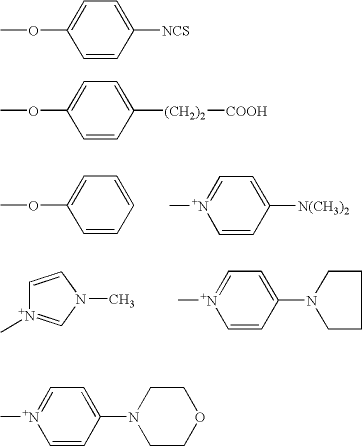
   the substituents R₄ in the allylic position possibly forming, with the polyethylenic chain, 1 to 3 fused rings containing from 4 to 14 atoms, which may or may not be saturated, said rings possibly containing one or more atoms of O, N and S, and possibly being optionally substituted with an oxo group.

Advantageously, the fluorescent entity according to the invention comprises a fluorophore which is covalently attached to the oligonucleotide, either directly or via a spacer arm.

This spacer arm may, for example, consist of a divalent organic radical chosen from linear or branched C₁-C₂₀ alkylene groups optionally containing one or more double bonds or triple bonds and/or optionally containing one or more hetero atoms, such as oxygen, nitrogen, sulfur, phosphorus, or one or more carbamoyl or carboxamido group(s); C₅-C₈ cycloalkylene groups and C₆-C₁₄ arylene groups, said alkylene, cycloalkylene or arylene groups optionally being substituted with alkyl, aryl or sulfonate groups, or chosen from the groups: 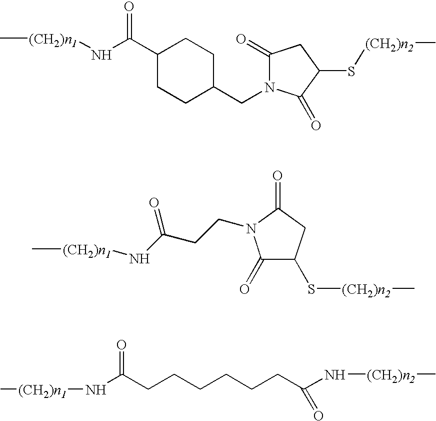
in which n₁ and n₂ are between 2 and 6.

According to a subsequent aspect, the invention also relates to the fluorescent conjugates consisting of an entity as defined above covalently attached to a carrier molecule.

Advantageous conjugates are those in which the final molar ratio, defined as the number of moles of fluorescent entities per carrier molecule, is greater than 0 and less than 100, preferably less than 20.

The carrier molecule is, for example, an antibody, an antigen, an intracellular messenger, an intercellular messenger, a protein, a peptide, a hapten, a lectin, biotin, avidin, streptavidin, a toxin, a carbohydrate, an oligosaccharide, a polysaccharide, a nucleic acid, a hormone, a vitamin, a medicinal product, a polymer, a polymeric particle, glass, a particle of glass or a surface made of glass or of a polymer.

The use of the fluorescent entities according to the invention makes it possible to produce conjugates exhibiting virtually zero aggregation of the fluorophore. Consequently, the quantum yield of the fluorophore can be almost completely conserved after attachment to carrier molecules, even when the final molar yield (number of moles of fluorescent entities per carrier molecule) increases.

This makes these conjugates very advantageous for use in a fluorescent system using nonradiative energy transfer (of the HTRF type). They are also of great advantage in more conventional techniques of detection by fluorescence, where the number of fluorophores per carrier molecule, the quantum yield and the molar extinction coefficient of the fluorophore are predominant criteria for the sensitivity of these systems.

The invention therefore also relates to the use of a fluorescent entity or of a fluorescent conjugate as defined above, as fluorescent tracer(s), for example for detecting and/or determining, by fluorescence, an analyte in a medium liable to contain it or for determining an interaction between biomolecules; or for determining a biological activity such as: an enzyme activity, the activation of a membrane-bound receptor, the transcription of a gene, a membrane transport or a variation in membrane polarization, in particular in a method for screening medicinal products.

The fluorescent conjugates according to the invention can be used as acceptor fluorescent compounds in the presence of donor fluorescent compounds or as donor fluorescent compounds in the presence of acceptor fluorescent compounds, in particular in fluorescence microscopy, in flow cytometry, in fluorescence polarization or in fluorescence correlation.

They can also be advantageously used as a contrast agent for optical imaging in vivo.

A subject of the invention is also a method for decreasing the phenomenon of aggregation at the surface of a carrier molecule attached to a fluorophore, characterized in that a fluorescent entity as defined above is used in place of said fluorophore.

Finally, a subject of the invention is a method for increasing the quantum yield of a fluorophore attached to a carrier molecule, characterized in that a fluorescent entity as defined above is used as a fluorophore.

The fluorescent entities according to the invention can be prepared as described below, by coupling “functionalized” oligonucleotides with a fluorophore.

In the present description, the term “functionalized oligonucleotide” is intended to mean an oligonucleotide comprising at least one chemically reactive function or a chemical group (such as a fluorescent group) which is not present in a natural oligonucleotide and which results from the incorporation of a modified nucleotide or of a non-nucleotide unit carrying this chemically reactive function or this chemical group. This chemically reactive function makes it possible, inter alia, to perform the synthesis of conjugates of oligonucleotides or of modified oligonucleotides. The terms “chemically reactive function”, “modified nucleotide”, “non-nucleotide unit” and “oligonucleotide conjugates” are understood to be in the sense described, for example, in the review by J. Goodchild [Conjugates of oligonucleotides and modified oligonucleotides: A review of their synthesis and properties. Bioconjugate chemistry, (1990) 1(3), 77-99]. The term “natural oligonucleotide” denotes a polynucleotide formed by the series of nucleotide units existing in nucleic acids [Abbreviations and symbols for the description of conformations of polynucleotide chains. Eur. J. Biochem. (1983) 131, 9-15].

The following examples illustrate the invention in a nonlimiting manner.

The following abbreviations are used:
DTT: dithiothreitol
DSS: N-(disuccinimidyl) suberate
GST: glutathione S-transferase
MOPS: 3-[N-morpholino]propanesulfonic acid
SPDP: N-succinimidyl-3-(2-pyridyldithio)propionate
SSMCC: 3-sulfo-N-hydroxysuccinimide ester of 4-(N-maleimidomethyl)cyclohexane-1-carboxylic acid
TEAB: triethylammonium bicarbonate
TEA Ac: triethylammonium acetate containing 10% of acetonitrile.
   I/Synthesis of Conjugates

### EXAMPLE 1

### Synthesis of the Compounds CY5-T15-hexylamine, CY5-T10-hexylamine and CY5-T5-hexylamine

This example describes the synthesis of an oligonucleotide of sequence T₅, T10 or T₁₅ functionalized at its 5′ end with a cyanin molecule such as CY5 which is nonsulfonated, and at its 3′ end with an arm carrying an amine group which can be used to label a biological molecule of interest. The general structure of the compound CY15-T10 hexylamine for example can be symbolized by ^{5′}(CY5-TTT TTT TTT T-hexylamine)₃.

The term “hexylamine” denotes an arm composed of 6 carbon atoms, possibly substituted and carrying an amine function.

In the present example, a 2-hydroxymethyl-6-aminohexanol arm is linked via a phosphate bridge formed between the hydroxyl in the 3′ position of the nucleotide located at the 3′ end and the 2-hydroxymethyl of the arm.

A solid support of the CPG (controlled pore glass) type conventionally used for synthesizing oligonucleotides is used. Such a support is referred to as “functionalized” since grafted onto the CPG is a chemical structure carrying a protected amine function capable, after final deprotection of the oligonucleotide, of releasing an aliphatic primary amine function.

A commercially available phosphoramidite derivative of thymidine is used for synthesizing the sequence T₁₅.

A commercially available phosphoramidite derivative of a nonsulfonated cyanin, which makes it possible to directly introduce the fluorescent marker such as cyanin (CY5) in the 5′ position of the oligonucleotide, is used.

The synthesis is carried out using an automatic DNA synthesizer (Applied Biosystems type 392) according to the manufacturer's protocol. The column containing the solid support (CPG) grafted (1 μmol) with a 2-O-dimethoxytrityl-6-fluorenylmethoxycarbonylaminohexane-1-succinoyl-long chain alkylamino-CPG derivative is placed on the synthesizer, the sequence T₁₅ is synthesized by performing fifteen cycles of synthesis using the phosphoramidite derivative of thymidine, and then a coupling cycle is carried out using the phosphoramidite derivative of the nonsulfonated cyanin (CY5).

At the end of this synthesis, the column is subjected to ammoniacal treatment (approximately 2 ml of 28% aqueous ammonia), making it possible to cleave the bond between the oligonucleotide and the CPG support, according to the manufacturer's protocol. The flask for collecting the released oligonucleotide is sealed, kept at 50-55° C. for 2 h, and then brought back to ambient temperature. The content of the flask (2 ml) is then transferred into a 5 ml polypropylene tube and then evaporated to dryness under vacuum using a speed-vac. The residue is then taken up with 500 μl of 10 mM TEAB. The solution obtained contains the “crude” oligonucleotide and predominantly the desired compound 5 (CY5-(T)₁₅-2-oxymethyl-6-aminohexanol)_{3′.}

The compound ^{5′}(CY5-(T)₁₅-2-oxymethyl-6-aminohexanol)₃, is obtained after HPLC purification on a LiChrospher RP-18^{e} 250-10 (10μ) column (Merck) using a gradient of acetonitrile in aqueous TEAAc (buffer A: 5% acetonitrile in 25 mM TEAAc, buffer B: 50% acetonitrile in 25 mM TEAAc; flow rate 5 ml/min, linear gradient of 10% B to 20% B in 20 min and a linear gradient of 20% to 100% of B in 10 min. The sequences which are incomplete and which do not comprise CY5 are eluted around 20 min, the fractions containing the desired sequence ^{5′}(CY5-(T)₁₅-2-oxymethyl-6-aminohexanol)₃, are collected around 28 min (these fractions are blue in color due to the presence of the CY5 group).

The fractions containing the desired sequence are pooled, and evaporated to dryness using a speed-vac, the residue being taken up with pure water. A UV/visible spectrum (210 nm to 750 nm) effected on a dilution of this solution makes it possible to determine the concentration of the oligonucleotide by its absorbence at 260 nm and to characterize the presence of the cyanin group (CY5) by its absorbence at 650 nm.

The compounds CY5-T10-hexylamine and CY5-T5-hexylamine are synthesized in the same way, by varying the number of cycles of synthesis in the automatic synthesizer.

### EXAMPLE 2

### Activation of the Compounds CY5-T15-hexyl-amine, CY5-T10-hexylamine and CY5-T5-hexylamine in the 3′ Position with SSMCC

The compound CY5-T15-hexylamine obtained in example 1 is dissolved in a 200 mM PO₄ buffer and the pH is adjusted to 8.

250 equivalents of SSMCC are added to the solution obtained. The reaction mixture is incubated for 30 min at ambient temperature, with stirring.

The compound CY5-T15-hexylamine activated with SSMCC, hereinafter referred to as CY5-T15-maleimide (hereinafter named CY5-T15 (mal)), is purified on an HR 10/30 G25 (SF) column in 10 mM PO₄ buffer containing 2 mM EDTA, pH 7. The purification is carried out at 60 ml/h.

The solution of CY5-T15-maleimide is concentrated by evaporation in the speed-vac.

The same procedure is carried out for CY5-T10-hexylamine.

For the activation of the compound CY5-T5-hexylamine, only 150 equivalents of SSMCC are used. An additional purification step on an HR 10/30 column is necessary.

### EXAMPLE 3

### Activation of CY5-T15-hexylamine in the 3′ Position with DSS(N-(disuccinimidyl suberate)

The compound CY5-T15-hexylamine obtained in example 1 is taken up in 10 μl of 100 mM MOPS buffer, pH 7.6, and 5 μl of acetonitrile.

150 equivalents of DSS (35 mg/ml in DMF) are added to the solution obtained.

The reaction mixture is incubated overnight at 4° C. with stirring. The compound CY5-T15-hexylamine activated with DSS, hereinafter referred to as CY5-T15-NHS, is purified on a NAP 5 G25 (SF) column in 5 mM MOPS buffer, pH 6.5.

The compound CY5-T15-NHS is then concentrated by several precipitations with butanol and centrifugations. The pellet is taken up in water.

The same procedure is carried out for the activation of the compounds CY5-T10-hexylamine and CY5-T5-hexylamine.

### EXAMPLE 4

### Preparation of the Conjugate CY5-(T)ₙ-hexyl-amine Activated with SSMCC (Example 2)—anti-GST Antibody (GSS11)

GSS11-T15-CY5 Batch 02B (mal):

The antibody GSS11 (CIS bio international, France) in 0.1 M carbonate buffer, pH 9, is activated by adding 8 equivalents of SPDP for 30 min at ambient temperature, with stirring, and then adding a final concentration of 20 mM of DTT for 15 min at ambient temperature, without stirring.

It is purified on an HR 10/10 G25 (SF) column in 0.1 M PO₄ buffer, pH 7.

The antibody thus activated is mixed with the oligonucleotide CY5-T15-maleimide obtained in example 2, with an initial CY5-T15-maleimide/antibody GSS11 molar ratio of 7.6. The incubation is from 18 to 20 h at +4° C.

The concentration of the antibody during the coupling is 0.5 mg/ml.

GSS11-T15-CY5 batch 03 (mal):

The antibody GSS11 is activated only by adding DTT at a concentration of 5 mM. The same procedure as for batch O₂B above is then carried out, with an initial CY5-T15-maleimide/antibody GSS11 molar ratio of 10.8.

The concentration of the antibody during the coupling is, in this case, 0.68 mg/ml.

GSS11-T5-CY5 Batch 01 (mal):

The same procedure as for batch O₂B above is carried out, with an initial CY5-T5-maleimide/antibody GSS11 molar ratio of 8.

The concentration of the antibody during the coupling is −0.9 mg/ml.

GSS1-T10-CY5 Batch 01 (mal):

The same procedure as for the GSS11-T5-CY5 batch 01 (mal) is carried out.

The coupling products are purified on an HR 10/30 Superdex 200 column at 60 ml/h.

The elution buffer is a 0.1 M phosphate buffer, pH 7.

The purifications are followed using a diode array detector (followed at various wavelengths).

### EXAMPLE 5

### Preparation of the Conjugate CY5-(T)ₙ-hexylamine Activated with DSS (Example 3)—anti-GST Antibody (GSS11)

GSS11-T15-CY5 Batch 01 (NHS):

The antibody GSS11 in 0.1 M carbonate buffer, pH 9, is mixed with the CY5-T15-NHS solution obtained in example 3. The initial CY5-T15-NHS/antibody molar ratio is 8. The concentration of the antibody during the coupling is 3.3 mg/ml. The incubation is for 30 min at ambient temperature without stirring.

GSS11-T15-CY5 Batch 02 (NHS):

The protocol is the same as for GSS11-T15-CY5 batch 01 NHS, with an initial CY5-T15-NHS/antibody molar ratio of 12. The incubation is for 2 h 30 at ambient temperature, but with stirring.

The coupling products are purified on an HR 10/30 Superdex 200 column at 60 ml/h. The elution buffer is a 0.1 M phosphate buffer, pH 7.

The purifications are followed using a diode array detector (followed at various wavelengths).

### EXAMPLE 6

### Synthesis of the Conjugate CY5sulfo-GSS11

These conjugates, which do not comprise any oligonucleotide, serve as reference compounds to show the advantages of the conjugates according to the invention.

A sulfonated cyanin is used here, and not a nonsulfonated cyanin as in the previous examples, since the quantum yield of the latter is virtually zero if it is not coupled to an oligonucleotide.

GSS11-CY5sulfo Batch M5:

Sulfonated cyanin (CY5sulfo) mono NHS is added to antibody GSS11 in 0.1 M carbonate buffer, pH 9.

The initial CY5sulfo/antibody molar ratio is 4.

The incubation is for 1 h at ambient temperature with stirring, the concentration of the antibody during the coupling is 5.2 mg/ml.

The purification is carried out on an HR10/10 G25 (SF) column in 0.1 M phosphate buffer, pH 7.

GSS11—CY5sulfo Batch M6:

The protocol is the same as for batch M5 above, but with an initial CY5sulfo/antibody molar ratio of 10.

GSS11—CY5sulfo Batch M7:

The protocol is the same as for batch M5 above, but with an initial CY5sulfo/antibody molar ratio of 20.

### EXAMPLE 7

### Preparation of the Conjugate CY5-A15-cAMP

The compound Cy5-A15-hexylamine is obtained according to a procedure similar to that described for the Cy5-T15-hexylamine in example 1, using a phosphoramidite derivative of adenosine in place of the phosphoramidite derivative of thymidine.

The synthesis of the N-hydroxysuccinimide ester of 2′-monosuccinyladenosine 3′,5′-cyclic monophosphate (cAMP-succ-NHS) is carried out as follows:

2 mg (3.9 μmol) of 2′-monosuccinyladenosine 3′,5′-cyclic monophosphate (sodium salt, Sigma # M9631) are suspended in 15 μl of a solution of N-hydroxysuccinimide (13.3 mg/ml in anhydrous DMSO), in an eppendorff tube.

35 μl of a solution of 1-(3-dimethylaminopropyl)-3-ethylcarbodiimide hydrochloride (200 mg/ml in anhydrous DMSO) are then added to the reaction volume and then placed on a shaker at ambient temperature.

After reaction for 90 minutes, the reaction mixture is diluted with DMSO qs 160 μl, so as to obtain a solution of [cAMP-succ-NHS]=23 nmol/μl, which is used for the synthesis of conjugates.

The cAMP-succ-NHS taken up in H₂O is then mixed with a solution of CY5-A15-hexylamine in 10 mM TEAB buffer, pH=8.5. During the coupling reaction, the molar ratio is 20 cAMP-NHS per CY5-A15-hexylamine. The cAMP concentration during the coupling is 5 μmol/ml. The incubation is for 2 h 30 at ambient temperature with agitation.

The purification is carried out using desalification on a NapS (G25) column in 0.1 M PO₄ buffer, pH 7.

II—Photophysical Properties

### EXAMPLE 8

### Comparison of the Quantum Yields and of the OD max/OD 604 nm Ratio

The quantum yield reflects the efficiency of the fluorescent entity in releasing the energy which it receives: the higher it is, the more efficient the fluorescent entity. These quantum yields are measured using a fluorimeter. The excitation is carried out at 600 nm, the fluorescence is measured at 615 to 750 nm. The area of the fluorescence spectra obtained is calculated and used to determine the quantum yields.

The final molar ratio (FMR) expresses the number of fluorophores covalently coupled to the protein.

The absorption spectra of the fluorescent entities make it possible to calculate the OD max/OD 604 nm ratio. This ratio reflects a possible phenomenon of aggregation of the fluorophores, for example CY5, at the surface of the labeled proteins, for example the antibody GSS11 or streptavidin.

The quantum yield and the OD max/OD 604 nm ratio are determined for various compounds synthesized according to the preceding examples.

8.1/The results given in table 1 below concern the reference conjugates (comprising no oligonucleotide) prepared in example 6 above.

TABLE 1

| | Final molar | ODₘₐₓ/ | Quantum |
|---|---|---|---|
| | ratio (FMR) | OD_{604 nm} | yield |
| CY5sulfo-mono NHS | — | 3.16 | 19% |
| GSS11-CY5sulfo batch M5 | 2.20 | 2.18 | 11% |
| GSS11-CY5sulfo batch M6 | 3.80 | 1.64 | 4% |
| GSS11-CY5sulfo batch M7 | 6.00 | 1.24 | 1% |

These results show that the coupling of the sulfonated cyanin CY5sulfo with an antibody GSS11 leads to a decrease in the quantum yield, associated in particular with a phenomenon of aggregation of CY5 at the surface of the antibody, as shown by the decrease in the ODmax/OD 604 nm ratio.

The more the number of CY5 molecules per antibody molecule (FMR) increases, the greater this decrease.

8.2/The results given in table 2 below concern the conjugates prepared in examples 4 and 5. The compounds CY5-T10-hexylamine and CY5-T15-hexylamine used as references are prepared as described in example 1.

TABLE 2

| | Final molar | ODₘₐₓ/ | Quantum |
|---|---|---|---|
| | ratio (FMR) | OD_{604 nm} | yield |
| CY5-T15-hexylamine | — | 2.79 | 25% |
| GSS11-T15-CY5 batch 02B (mal) | 4.30 | 2.59 | 19% |
| GSS11-T15-CY5 batch 03 (mal) | 1.40 | 2.59 | 18% |
| GSS11-T15-CY5 batch 01 (NHS) | 2.00 | 2.67 | 20% |
| GSS11-T15-CY5 batch 02 (NHS) | 5.10 | 2.63 | 20% |
| CY5-T10-hexylamine | — | 2.78 | 26% |
| GSS11-T10-CY5 batch 01 (mal) | 4.60 | 2.38 | 14% |

These results show that, when coupling the antibody GSS11 with fluorescent entities according to the invention, a very small decrease in the quantum yield and in the OD max/OD 604 nm ratio is observed, in comparison to the reference conjugate, for increasing FMRs. This clearly shows that the fluorescent entities according to the invention exhibit completely unexpected properties in terms of decreasing the aggregation at the surface of the labeled protein (here the antibody GSS11).

In addition, the result obtained is independent of the method of activation of the compound CY5-T15-hexylamine (DSS or SSMCC).

8.3/The results given in table 3 below concern the product prepared in example 7. The compound Cy5-A15-hexylamine used as a reference is prepared according to the procedure described for Cy5-T15-hexylamine in example 1.

TABLE 3

| | ODₘₐₓ/OD₆₀₄ | Quantum yield |
|---|---|---|
| CY5-A15-NH2 | 2.7 | 16% |
| CY5-A15-cAMP | 2.65 | 19.5% |

The results above show that the quantum yield of fluorescence of the fluorescent entities is slightly increased after coupling to the cyclic AMP. The conjugate thus created exhibits no sign of aggregation and thus has unexpected fluorescence properties.

### EXAMPLE 9

### Fluorescence Spectrum at Constant Antibody Concentration

In order to compare the level of fluorescence obtained with the various conjugates, fluorescence emission spectra (intensity of fluorescence as a function of the emission wavelength) were produced with a constant concentration of antibody (50 M). These spectra are produced on an LS50B spectrofluorimeter (PerkinElmer), with the settings as follows: excitation wavelength 600 nm, emission wavelength 615 to 750 nm, scanning rate 480 nm/min. Based on these spectra, it is possible to determine the intensity of fluorescence of the conjugate by calculating the area of the spectrum obtained by integration.

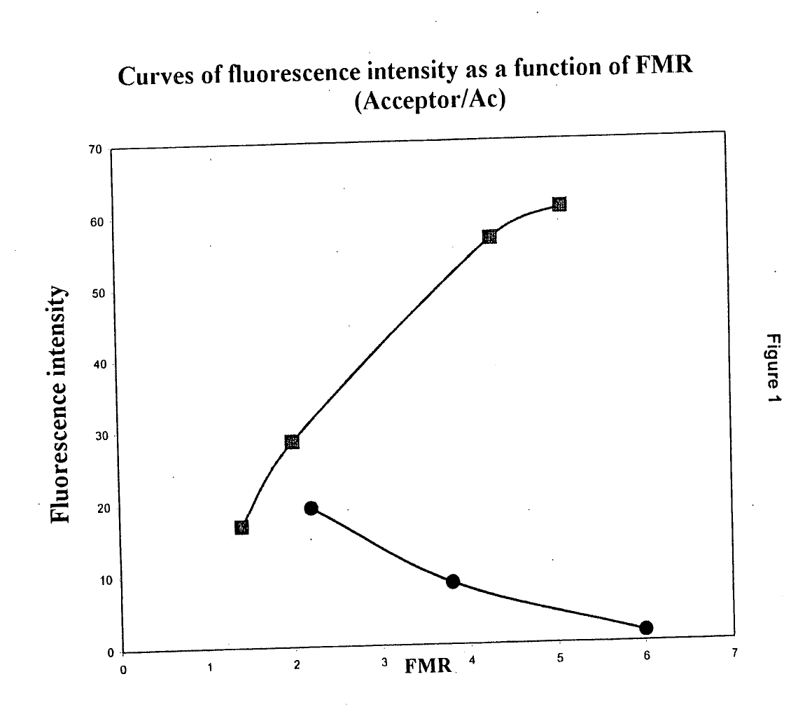

FIG. 1 gives the relationship between the FMR of the conjugates GSS11-CY5sulfo and GSS11-T15-CY5 and their intensity of fluorescence.

The following symbols are used:
—●— represents the conjugate GSS11-CY5 sulfo
—▪— represents the conjugate GSS11-T15-CY5

The graph in FIG. 1 shows that, in the case of the conjugates GSS11—CY5sulfo, the increase in the FMR of the conjugate leads to a decrease in its overall fluorescence, due to the extreme aggregation of the CY5 at the surface of the antibody. On the other hand, in the case of the conjugates GSS11-T15-CY5, the absence of aggregation of the CY5 makes it possible to maintain a high quantum yield and, consequently, to obtain an overall fluorescence which is virtually proportional to the number of CY5 per antibody.

III—Use of the Fluorescent Entities According to the Invention in Assays of the FRET (“Fluorescence Resonance Energy Transfer”) Type

### EXAMPLE 10

The fluorescent entities according to the invention can be used in systems of the FRET type well known to those skilled in the art.

In the present example, biotinylated Glutathione S-transferase (GST-biotin) is detected by measuring the fluorescence emitted by an acceptor compound, resulting from an energy transfer between a donor compound (conjugate europium cryptate-streptavidin (K(Eu)-Sa)) and an acceptor containing a fluorescent entity according to the invention (conjugate GSS11-oligonucleotide-CY5).

Assay Protocol:

The assay is carried out using a fluorimeter (Discovery, Packard), the excitation wavelength of which is 337 nm. The fluorescence is measured at 665 and 620 nm.
Assay buffer: 50 mM Hepes, pH 7, 0.1% BSA, 400 mM KF.
Reagents: GST-biotin, 20 nM solution
Donor: Sa-K(EU) NHS (CIS bio international)
Acceptor: GSS11-XL665 (CIS bio international) used as reference
   GSS11-T15-CY5 batch 02B mal (ex. 4) FMR 4.3
   GSS11-T15-CY5 batch 03mal (ex. 4) FMR 1.4
   GSS11-T15-CY5 batch 01NHS (ex. 5) FMR 2
   GSS11-T15-CY5 batch 02NHS (ex. 5) FMR 5.1
   GSS11-T10-CY5 batch 01mal (ex. 4) FMR 4.6
   GSS11-T15-CY5 batch 01mal (ex. 4) FMR 8.7

The following mixture is incubated for 20 h at ambient temperature:
50 μl of GST-biotin at 0-0.31-0.62-1.25-2.5 or 5 nM final concentration
100 μl of acceptor at 2.5 nM final concentration
50 μl of donor at 1 nM final concentration

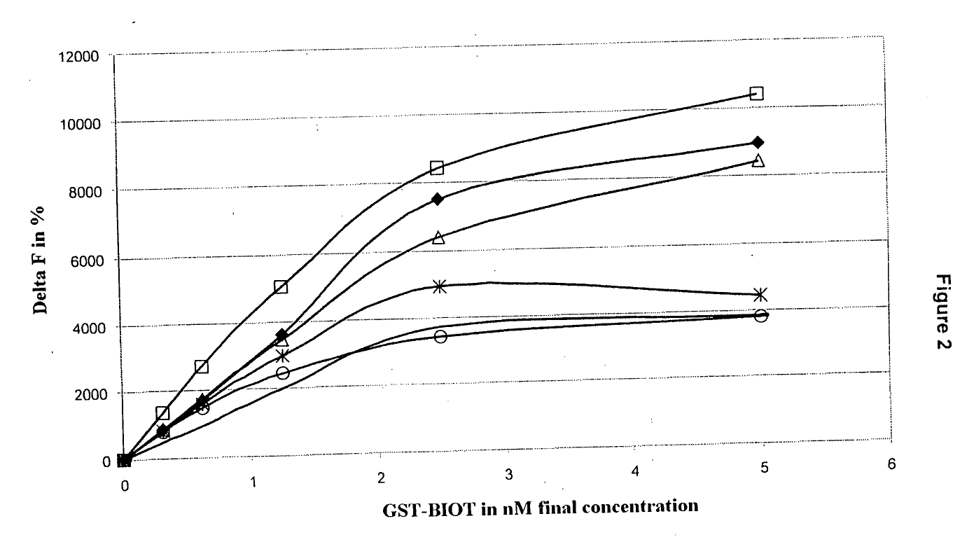

FIG. 2 represents the evolution of the signal (% delta F) as a function of the evolution of the concentration of the GST-biotin (GST-BIOT in nM final concentration).

The following symbols are used:
—∘— GSS11-XL-665
—♦— represents the conjugate GSS11-T15-CY5 batch O₂ NHS
— — represents the conjugate GSS11-T15-CY5 batch 01 NHS
—Δ— represents the conjugate GSS11-T15-CY5 batch 02B mal
—x— represents the conjugate GSS11-T15-CY5 batch 03 mal
— represents the conjugate GSS11-T10-CY5 batch 01 mal

The graph in FIG. 2 shows the advantage of the fluorescent entities according to the invention as fluorescent labels in an assay of the FRET type. Specifically, in all cases, the signal observed using the fluorescent entities according to the invention is greater than or equal to that obtained with the reference acceptor compound (XL).

### EXAMPLE 11

### Lifetime and Efficiency of Transfer of the Conjugates

The lifetimes and the efficiency of transfer obtained in the assay of the FRET type such as that of the preceding example were calculated. The conjugates tested were prepared as described in examples 4 and 5, the conjugate GSS11-XL665 serving as a reference.

The results are given in table 4 below.

TABLE 4

| | Sa-K batch 14 | | |
|---|---|---|---|
| | (NHS generic product) | | |
| | Lifetime | Lifetime of | |
| | of the | the free K | Efficiency of |
| | FRET in ms | in ms | transfer in % |
| | (τ_{FRET}) | (τ_{cryptate}) | 1 − (τ_{FRET}/τ_{cryptate}) |
| GSS11-T10-CY5 | 0.16 | 0.969 | 83% |
| batch 01mal | | | |
| GSS11-T5-CY5 | 0.21 | 1.030 | 80% |
| batch 01mal | | | |
| GSS11-T15-CY5 | 0.21 | 1.054 | 79% |
| batch 03mal | | | |
| GSS11-T5-CY5 | 0.16 | 1.001 | 83% |
| batch 02mal | | | |
| GSS11-T15-CY5 | 0.17 | 0.997 | 83% |
| batch 01NHS | | | |
| GSS11-T15-CY5 | 0.13 | 1.038 | 87% |
| batch 02NHS | | | |
| GSS11-XL665 | 0.27 | 1.033 | 73% |

The results show that the efficiency of energy transfer between the donor compound and the conjugates according to the invention used as acceptors is significantly higher for the conjugates according to the invention than for the conjugate XL665 (control).

### EXAMPLE 12

The fluorescent entities can be used in FRET competition systems well known to those skilled in the art.

In the present example, the presence of cyclic AMP (cAMP) in a sample is detected by observing the inhibition of the fluorescence energy transfer occurring between a donor compound (conjugate europium cryptate-anti-cAMP monoclonal antibody) and an acceptor compound containing a fluorescent entity (conjugate Cy5-A15-cAMP).

Assay Protocol:

The assay is carried out using a fluorimeter (Rubystar, BMG), the excitation wavelength of which is 337 nm. The fluorescence is measured at 665 nm and 620 nm.
Assay buffer: 0.1 M phosphate, pH=7, 0.1% BSA, 400 mM KF.
Reagents: cAMP, solution at 280 nM
Donor: conjugate Europium cryptate-anti-cAMP monoclonal antibody (anti-cAMP-K) (Cis bio international)
Acceptor:
conjugate Cy5-A15-cAMP (Cisbio international),
conjugate cAMP-XL665 used as a reference.

The following mixture is incubated for 20 h at ambient temperature:
25 μl buffer
25 μl cAMP at 0-0.07-0.27-1.09-4.37-17.5 or 70 nM final concentration
25 μl cAMP-XL665 or Cy5-A15-cAMP
25 μl anti-cAMP-K

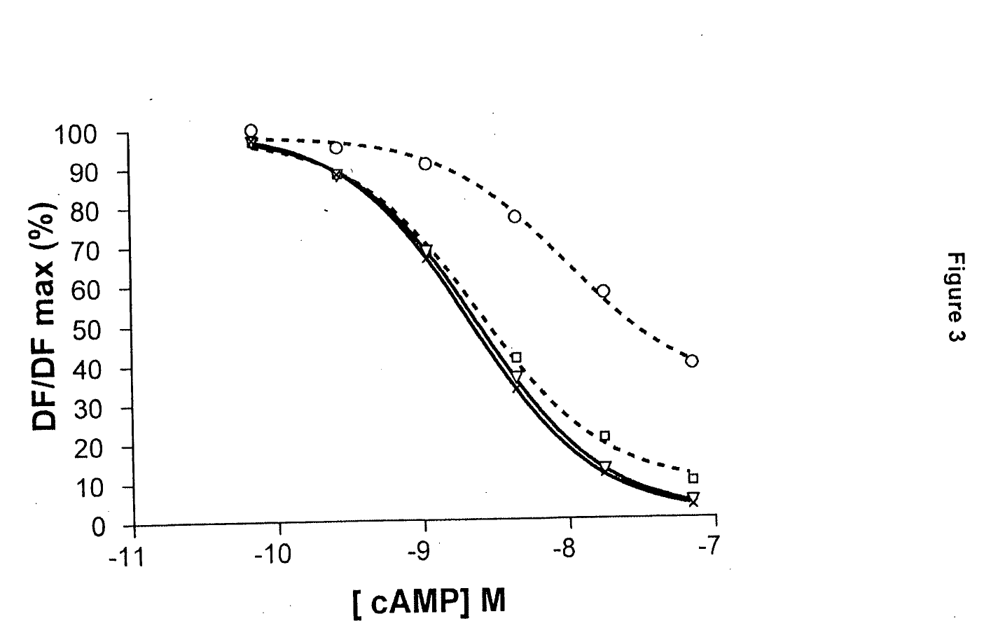

FIG. 3 represents the inhibition of FRET signal (DF/DF max) obtained at two incubation times (1 h and 20 h) in the presence of increasing amounts of cAMP.

The following symbols are used:
--- --- cAMP-XL665 incubation time 1H
---∘--- cAMP-XL665 incubation time 20H
—x— Cy5-T15-cAMP incubation time 1H
—∇— Cy5-T15-cAMP incubation time 20H

The graph in FIG. 3 shows the advantage of the fluorescent entities according to the invention as fluorescent labels in an assay of the FRET competition type. The sensitivity of the test is improved by using the fluorescent entities according to the invention, whatever the incubation time of the experiment. In addition, the loss of sensitivity observed with the reference acceptor (XL665) between 1 h and 20 h of incubation disappears when the fluorescent entities according to the invention are used.

1. A fluorescent entity comprising a fluorophore, with the exception of a rare earth metal cryptate, covalently attached to one or more oligonucleotide(s) or oligonucleotide analog(s), characterized in that it comprises at least one functional group, introduced or generated on the fluorophore or one of the oligonucleotides or oligonucleotide analogs.

2. The entity as claimed in claim 1, characterized in that the oligonucleotide or the oligonucleotide analog comprises from 2 to 60 nucleotide units.

3. The entity as claimed in claim 1, characterized in that the functional group can be attached to said entity via a spacer arm.

4. The entity as claimed in claim 1, characterized in that the fluorophore comprises one or more aromatic rings and has a high molecular extinction coefficient, greater than 20 000, preferably greater than 50 000.

5. The entity as claimed in claim 1, characterized in that the fluorophore is chosen from rhodamines, cyanins, squaraines, bodipys, fluoresceines and their derivatives.

6. The entity as claimed in claim 1, characterized in that the functional group is chosen from the groups: maleimide, carboxylic acid, haloacetamide, alkyl halide, azido, hydrazido, aldehyde, ketone, amino, sulfhydryl, isothiocyanate, isocyanate, monochlorotriazine, dichlorotriazine, aziridine, sulfonyl halide, acid halide, hydroxysuccinimide ester, hydroxysulfosuccinimide ester, imido ester, hydrazide, azidonitrophenyl, azidophenyl, azide, 3-(2-pyridyldithio)proprionamide and glyoxal, and groups of formula: 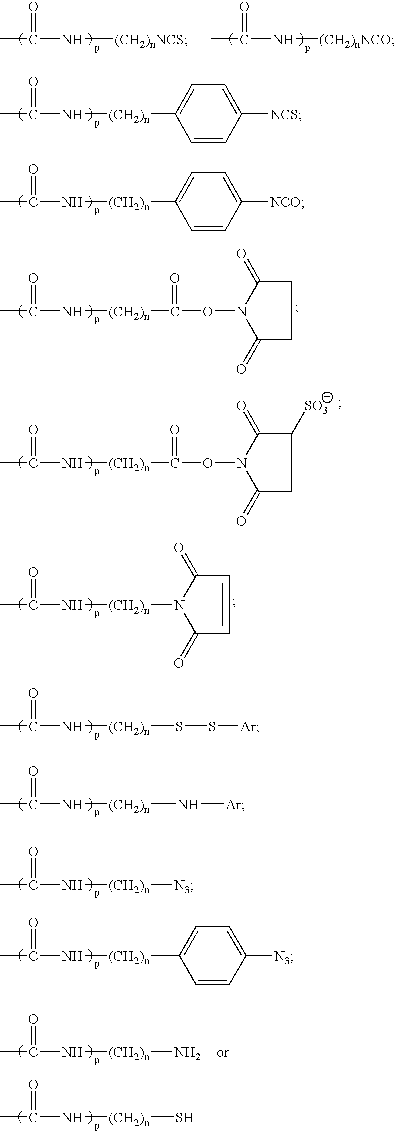
where n ranges from 0 to 8 and p is equal to 0 or 1, and Ar is a 5- or 6-membered heterocycle comprising 1 to 3 hetero atoms, optionally substituted with a halogen atom.

7. The fluorescent entity as claimed in claim 1, of formula (I): 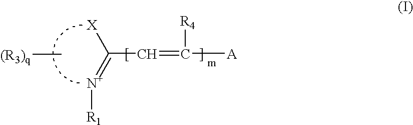
in which:
A represents a group chosen from: 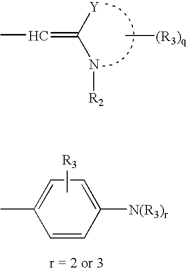
—N(R₃)ᵣ
r=2 or 3
the dashed lines each represent the carbon atoms required to form 1 to 3 fused rings, the groups R₃ being attached to these rings;
X and Y each represent N, C═O, O, S or C(CH₃)₂
m has a value 1, 2, 3 or 4;
q has a value 1, 2 or 3;
(R₃)_{q} represents q groups R₃, which may be identical or different;
the groups R₁, R₂ and R₃ are identical or different and are chosen from hydrogen; a group —(CH₂)ₛ-Z in which s ranges from 0 to 4 and Z represents a group CH₃, SO₃H, OH or N⁺R₁R₂R₃ in which R₁, R₂ and R₃ are as defined above; a functional group is chosen from the groups: maleimide, carboxylic acid, haloacetamide, alkyl halide, azido, hydrazido, aldehyde, ketone, amino, sulfhydryl, isothiocyanate, isocyanate, monochlorotriazine, dichlorotriazine, aziridine, sulfonyl halide, acid halide, hydroxy-succinimide ester, hydroxysulfosuccinimide ester, imido ester, hydrazide, azidonitrophenyl, azidophenyl, azide, 3-(2-pyridyldithio)proprionamide and glyoxal, and groups of formula: 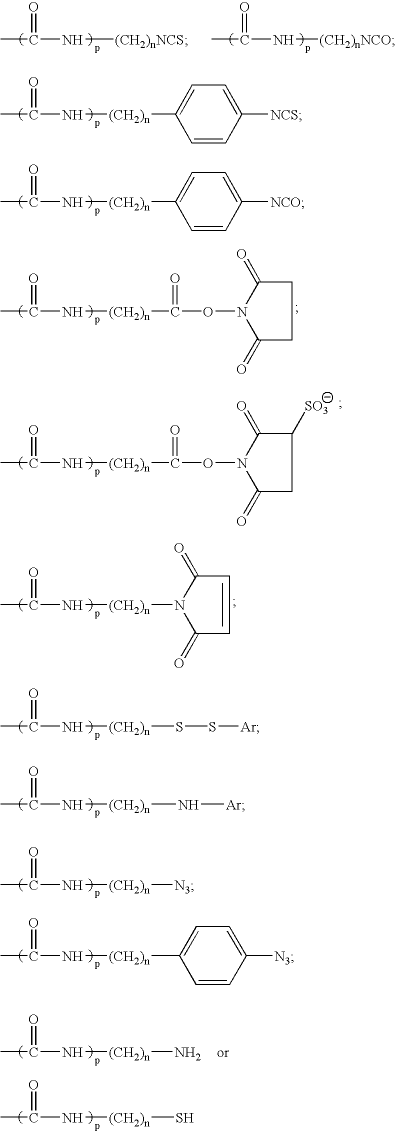
where n ranges from 0 to 8 and p is equal to 0 or 1 and Ar is a 5- or 6-membered heterocycle comprising 1 to 3 hetero atoms, optionally substituted with a halogen atom; and an oligonucleotide or oligonucleotide analog optionally comprising a functional group as said;
R₄ is chosen from: H; OH; CH₃; Cl and the groups of formula: 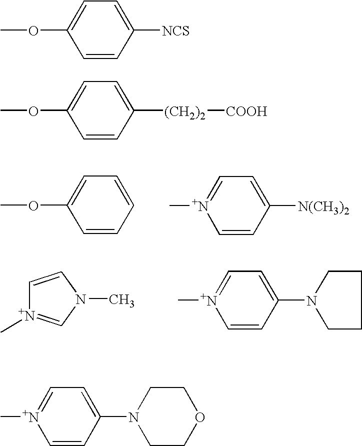
the substituents R₄ in the allylic position possibly forming, with the polyethylenic chain, 1 to 3 fused rings containing from 4 to 14 atoms, which may or may not be saturated, said rings possibly containing one or more atoms of O, N and S, and possibly being optionally substituted with an oxo group.

8. The fluorescent entity as claimed in claim 1, of formula (II) or (III): 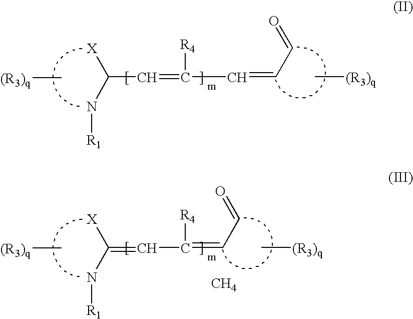
or
in which
the dashed lines each represent the carbon atoms required to form 1 to 3 fused rings, the groups R₃ being attached to these rings;
X represents N, C═O, O, S or C(CH₃)₂;
m has a value 1, 2, 3 or 4;
q has a value 1, 2 or 3;
(R₃)₂ represents q groups R₃, which may be identical or different;
the groups R₁ and R₃, which may be identical or different, are chosen from hydrogen; a group —(CH₂)ₛ-Z in which s ranges from 0 to 4 and Z represents a group CH₃, SO₃H, OH or N⁺R₁R₂R₃ in which R₁, R₂ and R₃ are as defined above; a functional group is chosen from the groups: maleimide, carboxylic acid, haloacetamide, alkyl halide, azido, hydrazido, aldehyde, ketone, amino, sulfhydryl, isothiocyanate, isocyanate, monochlorotriazine, dichlorotriazine, aziridine, sulfonyl halide, acid halide, hydroxy-succinimide ester, hydroxysulfosuccinimide ester, imido ester, hydrazide, azidonitrophenyl, azidophenyl, azide, 3-(2-pyridyldithio)proprionamide and glyoxal, and groups of formula: 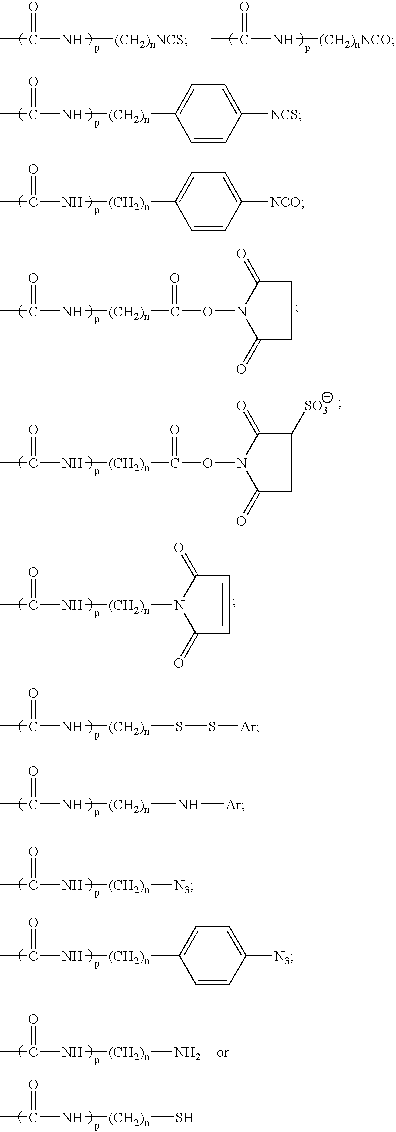
where n ranges from 0 to 8 and p is equal to 0 or 1, and Ar is a 5- or 6-membered heterocycle comprising 1 to 3 hetero atoms, optionally substituted with a halogen atom; an oligonucleotide or oligonucleotide analog optionally comprising a functional group as said;
R₄ is chosen from: H; OH; CH₃; Cl and the groups of formula: 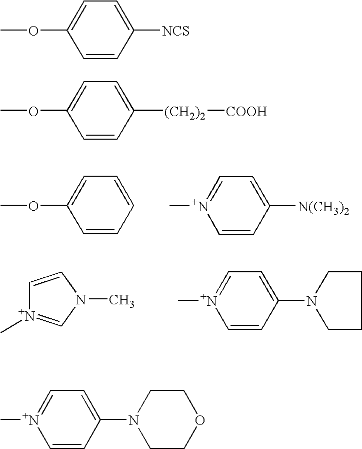
the substituents R₄ in the allylic position possibly forming, with the polyethylenic chain, 1 to 3 fused rings containing from 4 to 14 atoms, which may or may not be saturated, said rings possibly containing one or more atoms of O, N and S, and possibly being optionally substituted with an oxo group.

9. The fluorescent entity as claimed in claim 1, of formula (IV), (V), (VI) or (VII): 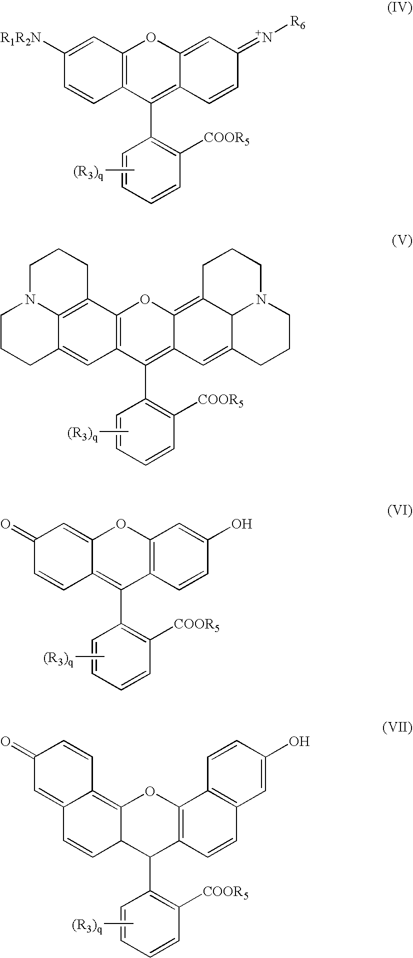
in which R₁, R₂, R₃ and R₅ are identical or different and are chosen from hydrogen; a group —(CH₂)ₛ-Z in which s ranges from 0 to 4 and Z represents a group CH₃, SO₃H, OH or N⁺R₁R₂R₃ in which R₁, R₂ and R₃ are as defined above; a functional group is chosen from the groups: maleimide, carboxylic acid, haloacetamide, alkyl halide, azido, hydrazido, aldehyde, ketone, amino, sulfhydryl, isothiocyanate, isocyanate, monochlorotriazine, dichlorotriazine, aziridine, sulfonyl halide, acid halide, hydroxy-succinimide ester, hydroxysulfosuccinimide ester, imido ester, hydrazide, azidonitrophenyl, azidophenyl, azide, 3-(2-pyridyldithio)proprionamide and glyoxal, and groups of formula: 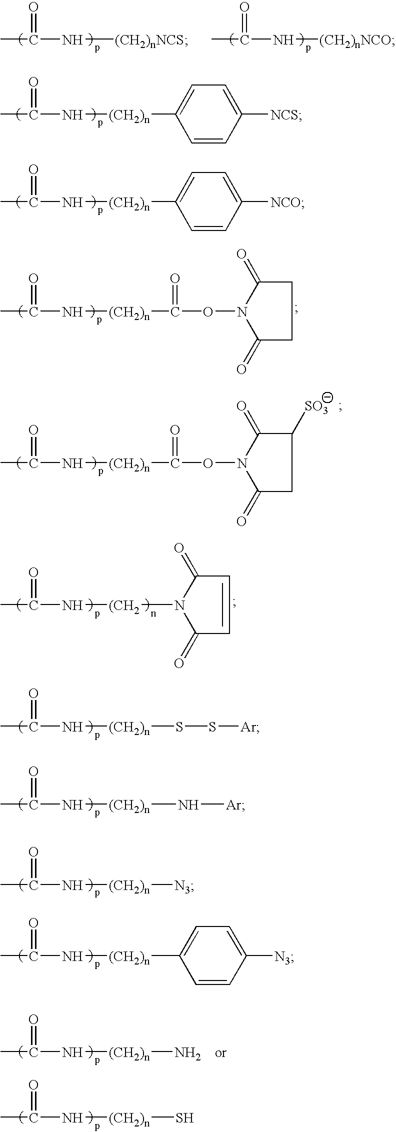
where n ranges from 0 to 8 and p is equal to 0 or 1, and Ar is a 5- or 6-membered heterocycle comprising 1 to 3 hetero atoms, optionally substituted with a halogen atom; and an oligonucleotide or oligonucleotide analog optionally comprising a functional group as said;
q has a value 1, 2 or 3.

10. The fluorescent entity as claimed in claim 1, of formula (VIII): 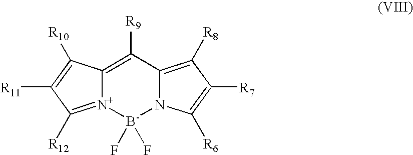
in which the substituents R₆ to R₁₂ are chosen from: hydrogen; a halogen; an alkyl; a cycloalkyl; aryl; arylalkyl; acyl; sulfo; a functional group is chosen from the groups: maleimide, carboxylic acid, haloacetamide, alkyl halide, azido, hydrazido, aldehyde, ketone, amino, sulfhydryl, isothiocyanate, isocyanate, monochlorotriazine, dichlorotriazine, aziridine, sulfonyl halide, acid halide, hydroxysuccinimide ester, hydroxysulfosuccinimide ester, imido ester, hydrazide, azidonitrophenyl, azidophenyl, azide, 3-(2-pyridyldithio)proprionamide and glyoxal, and groups of formula: 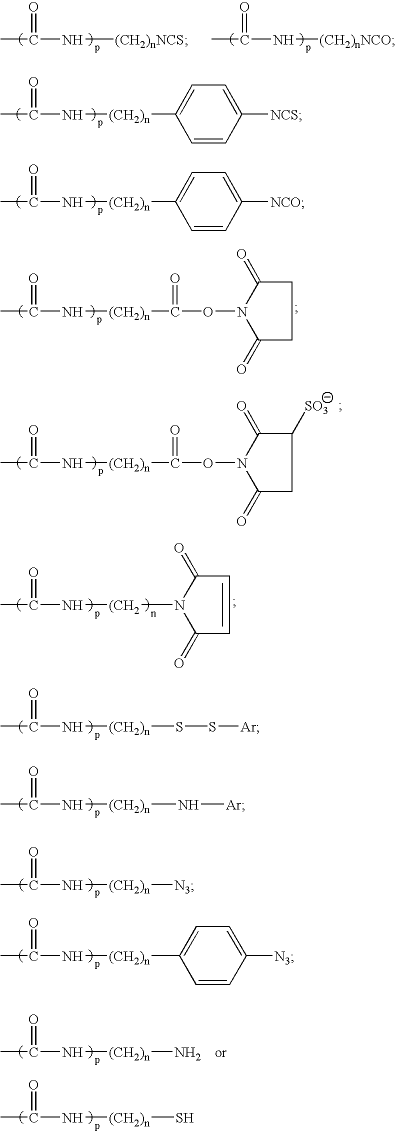
where n ranges from 0 to 8 and p is equal to 0 or 1 and Ar is a 5- or 6-membered heterocycle comprising 1 to 3 hetero atoms, optionally substituted with a halogen atom; and an oligonucleotide or oligonucleotide analog optionally comprising a functional group chosen from those mentioned as said.

11. The entity as claimed in claim 7, of formula (IX): 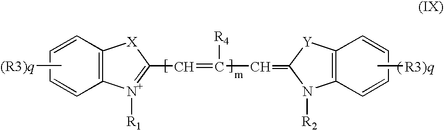
in which R₁, R₂, R₃, R₄, X, Y, m and q are as defined above.

12. The entity as claimed in claim 11, in which X and Y each represent a group C(CH₃)₂.

13. The entity as claimed in claim 11, in which
R₁ and R₂ represent an alkyl comprising from 1 to 4 carbon atoms or a group of formula below, at least one of the groups R₁ and R₂ representing a group of formula below: 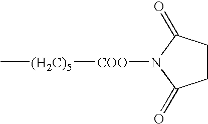
R₄ represents hydrogen
q=1, m=2
R₃ represents hydrogen; a group CH₂)ₛ-Z in which s ranges from 0 to 4 and Z represents a group CH₃, SO₃H, OH or N⁺R₁R₂R₃ in which R₁, R₂ and R₃ are as defined above; a functional group is chosen from the groups: maleimide, carboxylic acid, haloacetamide, alkyl halide, azido, hydrazido, aldehyde, ketone, amino, sulfhydryl, isothiocyanate, isocyanate, monochlorotriazine, dichlorotriazine, aziridine, sulfonyl halide, acid halide, hydroxysuccinimide ester, hydroxysulfosuccinimide ester, imido ester, hydrazide, azidonitrophenyl, azidophenyl, azide, 3-(2-pyridyldithio)proprionamide and glyoxal, and groups of formula: 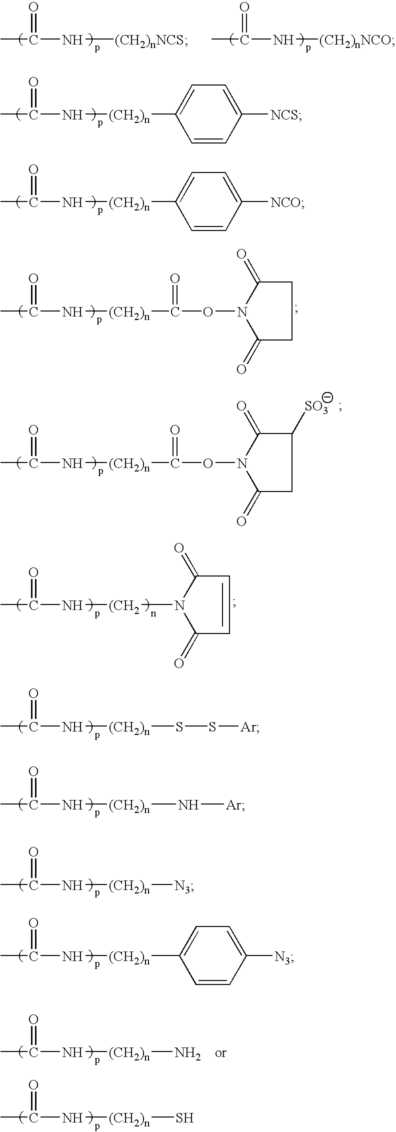
where n ranges from 0 to 8 and p is equal to 0 or 1, and Ar is a 5- or 6-membered heterocycle comprising 1 to 3 hetero atoms, optionally substituted with a halogen atom; an oligonucleotide or oligonucleotide analog optionally comprising a functional group as said;
R₄ is chosen from: H; OH; CH₃; Cl and the groups of formula: 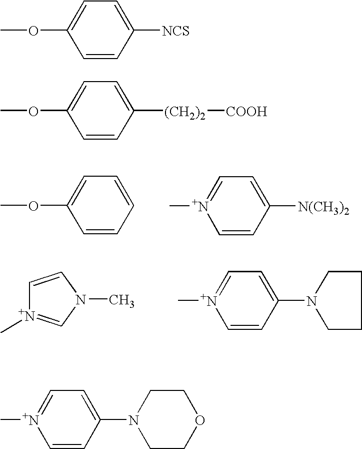
the substituents R₄ in the allylic position possibly forming, with the polyethylenic chain, 1 to 3 fused rings containing from 4 to 14 atoms, which may or may not be saturated, said rings possibly containing one or more atoms of O, N and S, and possibly being optionally substituted with an oxo group.

14. The entity as claimed in claim 1, characterized in that the fluorophore is covalently attached to the oligonucleotide either directly or via a spacer arm.

15. The entity as claimed in claim 14, characterized in that the fluorophore is attached to the oligonucleotide via a spacer arm consisting of a divalent organic radical chosen from linear or branched C₁-C₂₀ alkylene groups optionally containing one or more double bonds or triple bonds and/or optionally containing one or more hetero atoms, such as oxygen, nitrogen, sulfur, phosphorus, or one or more carbamoyl or carboxamido group(s); C₅-C₈ cycloalkylene groups and C₆-C₁₄ arylene groups, said alkylene, cycloalkylene or arylene groups optionally being substituted with alkyl, aryl or sulfonate groups.

16. The entity as claimed in claim 15, characterized in that the spacer arm is chosen from the groups: 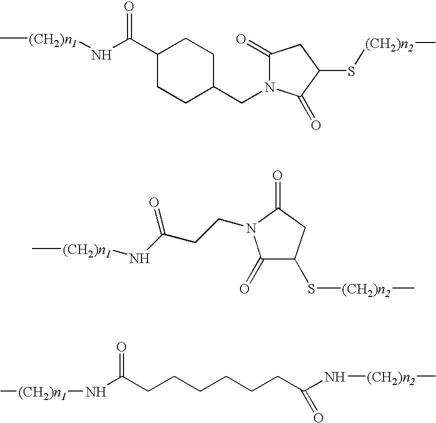
in which n₁ and n₂ are between 2 and 6.

17. The entity as claimed in claim 1, characterized in that the oligonucleotide comprises from 5 to 60, in particular 5 to 20, preferably from 5 to 15, nucleotide units.

18. The entity as claimed in claim 17, characterized in that the oligonucleotide consists of a series of ribonucleotide or deoxyribonucleotide units attached to one another via bonds of the phosphodiester type.

19. The entity as claimed in claim 17, characterized in that the oligonucleotide consists of a series of ribonucleotide or deoxyribonucleotide units or of nucleotide analog units modified on the sugar or on the base, attached to one another by natural internucleotide bonds of the phosphodiester type, some of the internucleotide bonds being optionally replaced with phosphonate, phosphoramide or phosphorothioate bonds.

20. The entity as claimed in claim 17, characterized in that the oligonucleotide consists of a series comprising both ribonucleotide or deoxyribonucleotide units attached to one another by bonds of the phosphodiester type and nucleoside analog units attached to one another by amide bonds.

21. The entity as claimed in claim 17, characterized in that the oligonucleotide consists of a series of ribonucleotide or deoxyribonucleotide units attached to one another by bonds of the phosphodiester type and of nucleoside analog units attached to one another by amide bonds, said oligonucleotide comprising at least 5 internucleotide bonds of the phosphodiester type at the end intended to be attached to the fluorophore.

22. The entity as claimed in claim 1, characterized in that the functional group is an amine function of a nucleotide unit of the oligonucleotide or of the oligonucleotide analog, or results from the reaction of a free amine function of a nucleotide unit of the oligonucleotide or the oligonucleotide analog, with a group chosen from the groups: ester, carboxylic acid, isothiocyanate, aldehyde, carbonyl, sulfonyl halide, alkyl halide, azide, hydrazide, dichlorotriazine, anhydride, haloacetamide, maleimide and sulfhydryl.

23. The entity as claimed in claim 1, characterized in that the functional group results from the reaction of a free amine function of a nucleotide unit of the oligonucleotide or of the oligonucleotide analog, with an N-hydroxysuccinimidyl ester.

24. The entity as claimed in claim 1, characterized in that the functional group(s) is (are) attached to the fluorophore and/or to the oligonucleotide by a spacer arm consisting of a divalent organic radical, chosen from linear or branched C₁-C₂₀ alkylene groups optionally containing one or more double bonds or triple bonds and/or optionally containing one or more hetero atoms, such as oxygen, nitrogen, sulfur, phosphorus, or one or more carbamoyl or carboxamido group(s); C₅-C₈ cycloalkylene groups and C₆-C₁₄ arylene groups, said alkylene, cycloalkylene or arylene groups being optionally substituted with alkyl, aryl or sulfonate groups.

25. The entity as claimed in claim 24, characterized in that the spacer arm is chosen from the groups: 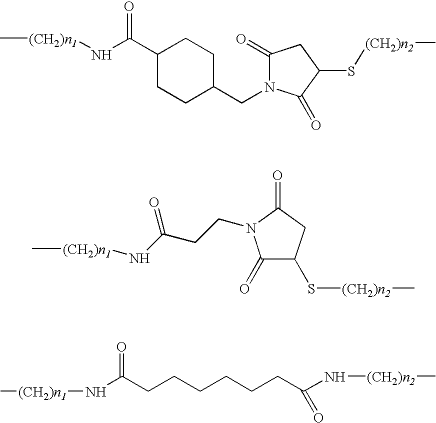
in which n, and n₂ are between 2 and 6.

26. A fluorescent conjugate consisting of an entity as claimed in claim 1 covalently attached to a carrier molecule.

27. The conjugate as claimed in claim 26, characterized in that the fluorophore of the fluorescent entity is cyanin-5, the oligonucleotide of said entity has the sequence A₁₅ and the carrier molecule is cAMP.

28. The conjugate as claimed in claim 26, characterized in that the final molar ratio is greater than 0 and less than 100, preferably greater than 0 and less than 20.

29. The fluorescent conjugate as claimed in claim 26, characterized in that the carrier molecule is an antibody, an antigen, an intracellular messenger, an intercellular messenger, a protein, a peptide, a hapten, a lectin, biotin, avidin, streptavidin, a toxin, a carbohydrate, an oligosaccharide, a polysaccharide, a nucleic acid, a hormone, a vitamin, a medicinal product, a polymer, a polymeric particle, glass, a particle of glass or a surface made of glass or of a polymer.

30. The fluorescent conjugate as claimed in claim 29, characterized in that the carrier molecule is an antibody or streptavidin.

31. The use of a fluorescent entity as claimed in claim 1, as a fluorescent tracer.

32. The use as claimed in claim 31, for detecting and/or determining, by fluorescence, an analyte in a medium liable to contain it.

33. The use as claimed in claim 32, for determining an interaction between biomolecules; or for determining a biological activity such as: an enzyme activity, the activation of a membrane-bound receptor, the transcription of a gene, a membrane transport or a variation in membrane polarization.

34. The use as claimed in claim 31, in a method for screening medicinal products.

35. The use as claimed in claim 34, in which a fluorescent conjugate is used as an acceptor fluorescent compound in the presence of a donor fluorescent compound.

36. The use as claimed in claim 35, in which a fluorescent conjugate is used as a donor fluorescent compound in the presence of an acceptor fluorescent compound.

37. The use as claimed in claim 35, in fluorescence microscopy, in flow cytometry, in fluorescence polarization or in fluorescence correlation.

38. The use of a conjugate as claimed in claim 26, as a contrast agent for optical imaging in vivo.

39. A method for increasing the fluorescence intensity of a fluorophore attached to a carrier molecule, characterized in that a fluorescent entity as claimed in claim 1 is used as a fluorophore.

40. A method for decreasing the phenomenon of aggregation at the surface of a carrier molecule attached to a fluorophore, characterized in that a fluorescent entity as claimed in claim 1 is used in place of said fluorophore.

41. A method for increasing the quantum yield of a fluorophore attached to a carrier molecule, characterized in that a fluorescent entity as claimed in claim 1 is used as a fluorophore.
